(19) United States
(12) Patent Application Publication
(10) Pub. No.: US 2026/0317695 A1
(43) Pub. Date: Oct. 15, 2026

(54) COATING FOR RETAINING CELLULAR CONTENTS, ENCAPSULATION METHOD, AND APPLICATION THEREOF

(71) Applicant: Novastra Therapeutics Inc., Dover, DE (US)

(72) Inventors: Junling GUO, Suzhou (CN); Yunxiang HE, Suzhou (CN)

(73) Assignee: Novastra Therapeutics Inc., Dover, DE (US)

(21) Appl. No.: 19/553,324

(22) Filed: Feb. 28, 2026

Related U.S. Application Data
(63) Continuation of application No. PCT/CN2025/107840, filed on Jul. 10, 2025.

(30) Foreign Application Priority Data
Apr. 11, 2025 (CN) 202510452174.X

(51) Int. Cl. C12N 5/00; B01J 13/10; B82Y 5/00; B82Y 40/00; C12N 1/20

(52) U.S. Cl. CPC C12N 5/0012; B01J 13/10; C12N 1/20; B82Y 5/00; B82Y 40/00

(57) ABSTRACT

A nanocoating for retaining cellular contents, an encapsulation method, and applications thereof are disclosed. The raw materials of the nanocoating include metal ions and plant polyphenols, which form metal-polyphenol nanoparticles under acidic conditions. After binding to a cell, the metal-polyphenol nanoparticles form a nanocoating adhering to the cell surface under alkaline conditions. The nanocoating of the present invention prevents the leakage of critical intracellular substances to maintain cellular structural integrity. Furthermore, the nanocoating is configured to prevent the leakage of harmful substances even after cell rupture, thereby mitigating risks associated with content leakage.

### CROSS-REFERENCE TO RELATED APPLICATIONS

This application is a National Stage Entry of PCT Application No. PCT/CN2025/107840, filed on Jul. 10, 2025, which in turn claims priority to Chinese Patent Application No. 202510452174.X, filed on Apr. 11, 2025, the entire contents of each of which are hereby incorporated by reference.

### TECHNICAL FIELD

The present invention relates to the field of biotechnology, and more particularly to a coating for retaining cellular contents, an encapsulation method, and applications thereof.

### BACKGROUND

In the biomedical and biotechnological fields, the preservation of the cell content is critical for maintaining the cell function and for ensuring the safety of the application (e.g. treatment, detection). However, leakage of intracellular proteins, nucleic acids, metabolites and organelle components is often caused by membrane integrity disruption under a variety of conditions, such as in vitro storage, mechanical stress (e.g., centrifugation, freeze thawing), pathological conditions (e.g., membrane damage), or specific application scenarios (e.g., transport of cell therapeutic products). Such leakage may cause various negative effects. For example, in stem cell therapy, loss of cell-secreted growth factors and cytokines can significantly reduce the therapeutic effect. In single cell sequencing technology, leakage of intracellular RNA can lead to cross-contamination between samples, affecting data reliability. In addition, for bacterial cells, leakage of the contents (e.g., endotoxin release) may also raise safety concerns.

In order to solve the problem of leakage of cell contents, various packaging techniques have been developed in recent years. The microencapsulation technology based on the alginic acid system mainly utilizes ion crosslinking to form a porous gel layer to wrap cells to protect the cells and reduce leakage of contents, but researches show that the pore diameter of calcium alginate gel is usually 50-200 nm and is far larger than the molecular sizes of proteins (molecular weight 10-500 kDa) and nucleic acids, so that the macromolecular substances are easy to diffuse to the external environment. The strategy for constructing the bionic cell membrane by using lipid double-layer modification is to strengthen the stability of the cell membrane by liposome fusion or membrane protein insertion so as to reduce the leakage of contents, but the method is faced with the problems of low modification efficiency and poor long-term stability in practical application, and a lipid coating is easy to fall off due to oxidation or mechanical stress. Chemical cross-linking methods, which strengthen the cell membrane structure by cross-linking agents in an attempt to prevent leakage of the contents, often require severe conditions such as low pH or high temperature, irreversibly disrupt the conformation of the cell membrane proteins, resulting in denaturation or loss of function of the contents, which limits the technology to applications where retention of the functions of the contents is required.

Therefore, developing packaging techniques that can effectively prevent leakage of cellular contents remains a critical challenge to be addressed.

### SUMMARY

It is an object of the present invention to provide a coating for retaining cellular contents. The coating comprises a metal-polyphenol complex system formed by metal ions and plant polyphenols. The coating is configured to adhere to a cell surface to effectively prevent leakage of cellular contents, maintain the structural integrity of the cell, and mitigate risks associated with content leakage.

According to one aspect of the present invention, a coating for retaining cellular contents is provided. The raw materials of the coating include metal ions and plant polyphenols. The metal ions and plant polyphenols are configured to form metal-polyphenol nanoparticles under acidic conditions. After the metal-polyphenol nanoparticles bind to a cell, they form a dense coating adhering to the cell surface under alkaline conditions.

In the present invention, the metal ions and plant polyphenols are mixed and the pH of the solution is adjusted to an acidic range. Under acidic conditions, the complexation between the plant polyphenols and the metal ions is incomplete, resulting in the formation of metal-polyphenol nanoparticles with a significantly small size and relatively weak inter-particle connections. Once these small-sized nanoparticles bind to the cell surface, the pH is adjusted to an alkaline range. Under alkaline conditions, the connection between individual nanoparticles, as well as between the nanoparticles and the cell surface, becomes more robust and compact, thereby forming a dense coating with extremely small interstitial gaps on the cell surface.

The interstitial gaps between the metal-polyphenol nanoparticles in the coating are sufficiently small to effectively block the passage of biological macromolecules such as proteins and nucleic acids. Furthermore, the phenolic hydroxyl groups abundant in the metal-polyphenol nanoparticles are capable of forming stable bonds with macromolecules (e.g., proteins and nucleic acids) through hydrogen bonding, hydrophobic interactions, or π-π stacking. This mechanisms allows the coating to anchor these substances within the interior of the encapsulated cell, preventing leakage. Consequently, the coating not only prevents the leakage of critical intracellular substances to maintain structural integrity, but also retains harmful intracellular substances even after cell rupture, thereby reducing potential safety risks. Additionally, the small gaps of the coating physically obstruct cellular proliferation and metabolic pathways, achieving cell inactivation. Thus, the coating serves the dual function of cell inactivation and leakage prevention.

In certain preferred embodiments, the plant polyphenols are selected from the group consisting of epigallocatechin gallate (EGCG), proanthocyanidins (PC), tara tannin (Tara), and tannic acid (TA). The metal ions are selected from the group consisting of zinc ions, manganese ions, and iron ions.

In a further embodiment, the raw materials of the coating include a stabilizer. The stabilizer is configured to react with the metal ions and plant polyphenols under acidic conditions to form the metal-polyphenol nanoparticles. The stabilizer improves the stability of the small-sized nanoparticles, facilitating their subsequent connection to the cells. Preferably, the stabilizer is selected from the group consisting of hyaluronic acid, chitosan, carboxymethyl cellulose, alginate, and alginate salts.

In another embodiment, the acidic conditions comprise a pH value of 3.5 to 5.5, and the alkaline conditions comprise a pH value of 7.0 to 9.0. Buffer solutions may be employed to adjust the pH, including but not limited to acetate buffer, citrate buffer, phosphate buffer, succinate buffer, Tris buffer, or borate buffer.

In a further embodiment, the molar ratio of the metal ions to the plant polyphenols is from 4:1 to 2:1. This specific range ensures the formation of stable, small-sized nanoparticles. If the metal ion content is too low, the coating may be incomplete or unstable; if the metal ion content is too high, the nanoparticle size increases, leading to larger interstitial gaps that reduce the anti-leakage efficacy. The diameter of the metal-polyphenol nanoparticles is preferably controlled within a range of 5 nm to 50 nm.

According to another aspect of the present invention, a method for encapsulating cells to retain cellular contents is provided, comprising:

Mixing a plant polyphenol solution and a metal ion solution to obtain a first solution;

Adding a stabilizer to the first solution and adjusting the first solution to an acidic pH to obtain a metal-polyphenol nanoparticle suspension; and

Adding the nanoparticle suspension to a cell suspension containing target cells to obtain a second solution, and adjusting the second solution to an alkaline pH to form a coating on the surface of the target cells.

The present invention further provides applications of the aforementioned coating for encapsulating stem cells, tumor cells, and bacterial cells. In stem cell therapy, the coating prevents the leakage of growth factors and cytokines (e.g., TGF-β, IGF-1, VEGF), thereby maintaining therapeutic potential. In bacterial applications, such as vaccine preparation, the coating retains endotoxins within the cell even after rupture, significantly reducing safety risks. The technology is applicable to various cells, including mammalian cells (tumor cells, immune cells, red blood cells) and bacteria (e.g., E. coli, S. aureus, L. casei, B. bifidum).

Advantages of the present invention include:

High-efficiency retention of macromolecules via physical barriers and chemical adhesion;

Controlled two-step pH adjustment for precise formation of dense coatings;

Optimized molar ratios for ideal nanoparticle size (5-50 nm);

Simplified operational procedures without the need for complex equipment;

Enhanced safety for bacterial-derived products by sequestering endotoxins;

Broad applicability across diverse cell types and biotechnological fields, including single-cell sequencing.

### BRIEF DESCRIPTION OF THE DRAWINGS

FIG. 1 is a flowchart of the encapsulation method according to an embodiment of the present invention.

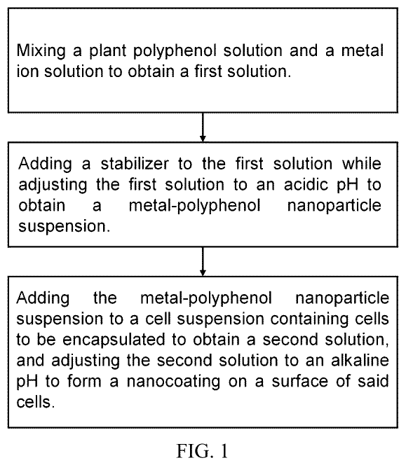

FIG. 2 shows Transmission Electron Microscopy (TEM) characterization of an untreated stem cell surface versus an EGCG-Mn encapsulated stem cell (C1) surface.

FIG. 3 illustrates stem cells (C1) labeled with FITC-BSA to confirm the presence of the coating.

FIG. 4 shows electrophoresis results of sediments from ruptured untreated stem cells (C1) versus encapsulated stem cells.

FIG. 5 shows the protein and RNA content collected from the sediment of ruptured encapsulated stem cells.

FIG. 6 shows E. coli cell (C2) labeled with an anthocyanin near-infrared fluorescent dye in an embodiment of the invention;

FIG. 7 shows electrophoresis results of endotoxin protein groups from ruptured E. coli cells (C2).

FIG. 8 shows protein and endotoxin content in the supernatant of ruptured E. coli cells (C2).

FIG. 9 shows a comparison of tumor cell activity before and after inactivation using Calcein/PI staining.

### DETAILED DESCRIPTION OF THE EMBODIMENTS

The following detailed description, in conjunction with the embodiments and accompanying drawings, is provided to facilitate a clearer understanding of the objectives, technical solutions, and advantages of the present invention. The exemplary embodiments described herein are intended to illustrate the present invention and should not be construed as limiting its scope.

The raw materials used in the present invention are not particularly limited in terms of source and may be purchased commercially or prepared according to conventional methods known to those skilled in the art. There are no specific restrictions on the purity of the raw materials; analytical grade purity or purity levels conventional in the field of biotechnology are preferred. All raw materials, including their grades and abbreviations, are conventional in the art and are clearly defined within their respective fields of application. A person skilled in the art can obtain these materials through commercial purchase or conventional preparation based on their grades, abbreviations, and corresponding uses.

Terms such as “first” and “second” (e.g., first solution, second solution, first buffer, second buffer, etc.) are used herein solely for clarity to distinguish between various components and are not intended to impose any specific order or imply importance. Furthermore, unless otherwise specified, the term “connection” as used herein may refer to either a direct connection or an indirect connection through other groups.

### I. Formation of Coating on Cell Surfaces

Current metal-polyphenol particles are primarily utilized for cell delivery, such as probiotic delivery, where maintaining cell viability and function during delivery is essential. In such conventional methods, polyphenols are first connected to the cells, and then metal ions are combined with the polyphenols to form a coating. Such coatings typically possess large interstitial gaps, which the cells utilize to perform their functions and maintain activity.

In contrast, the encapsulation method of the present invention is based on first forming loose, fine metal-polyphenol nanoparticles in a low-pH environment. After these nanoparticles are connected to the cells, the pH is adjusted to a higher value to form a dense nanocoating on the cell surface. The small interstitial gaps of the nanocoating particles prevent the leakage of cellular contents, thereby maintaining the functional and structural integrity of cells such as stem cells while mitigating risks associated with content leakage in cells such as bacterial cells. Furthermore, the inventors have discovered that during encapsulation, the nanocoating physically obstructs cellular proliferation and metabolic pathways, thereby achieving cell inactivation. This dual functionality significantly enhances the convenience and safety of related cellular experiments.

### Example 1: Preparation of Stem Cells Encapsulated with EGCG-Mn Nanocoating

Step 1 (S1): An epigallocatechin gallate (EGCG) solution (200 μL, 150 mg/mL) was dispersed in 1 mL of physiological saline, followed by the addition of a manganese chloride (MnCl₂ solution (40 μL, 150 mg/mL). The mixture was shaken at 100 rpm for 5 min to obtain a first solution.

Step 2 (S2): A hyaluronic acid solution (1 mg/mL) and a first phosphate buffer (pH=5.0, 20 mM, 1 mL) were added to the first solution. The mixture was shaken at 100 rpm for 5 min to obtain an EGCG-Mn nanoparticle suspension.

Step 3 (S3): Pretreated stem cells (2×10⁵ cells) were resuspended in physiological saline (0.9% sodium chloride solution, 1 mL). Subsequently, 400 μL of the EGCG-Mn nanoparticle suspension was added to the cell suspension, and the mixture was shaken at 200 rpm for 5 min to obtain a second solution.

Step 4 (S4): A second phosphate buffer (pH=8, 20 mM, 500 μL) was added to the second solution and shaken at 200 rpm for 5 min to yield stem cells (C1) encapsulated with a nanocoating composed of hyaluronic acid (HA)-embedded EGCG-Mn nanoparticles.

### Example 2: Preparation of E. coli Encapsulated with EGCG-Mn Nanocoating

S1-S2: The procedures were identical to S1 and S2 of Example 1 to obtain an EGCG-Mn nanoparticle suspension.

S3: Pretreated E. coli cells (1×10⁷ cells/mL, OD₆₀₀ pprox. 0.1) were resuspended in 1 mL of physiological saline. Then, 400 μL of the EGCG-Mn nanoparticle suspension was added and shaken at 200 rpm for 5 min.

S4: A second phosphate buffer (pH=8, 20 mM, 500 μL) was added and shaken at 200 rpm for 5 min to obtain E. coli cells (C2) encapsulated with the HA-embedded EGCG-Mn nanocoating.

### Example 3: Preparation of Tumor Cells Encapsulated with TA-Fe Nanocoating

S1: A tannic acid (TA) solution (100 μL, 250 mg/mL) was dispersed in 1 mL of saline, followed by the addition of a ferric chloride (FeCl³) solution (50 μL, 100 mg/mL) and shaken at 100 rpm for 5 min.

S2: A chitosan solution (1 mg/mL) and a first phosphate buffer (pH=5.0, 20 mM, 1 mL) were added and shaken at 100 rpm for 5 min to obtain a TA-Fe nanoparticle suspension.

S3: Pretreated B16F10 tumor cells (2×10⁵ cells) were resuspended in 1 mL of saline, mixed with 400 μL of the TA-Fe nanoparticle suspension, and shaken at 200 rpm for 5 min.

S4: A second phosphate buffer (pH=8.0, 20 mM, 500 μL) was added and shaken at 200 rpm for 5 min to obtain tumor cells (C3) encapsulated with the TA-Fe nanocoating.

### Example 4: Preparation of Tumor Cells Encapsulated with PC-Zn Nanocoating

S1-S2: A proanthocyanidin (PC) solution (200 μL, 150 mg/mL) and zinc chloride (ZnCl₂ solution (40 μL, 150 mg/mL) were used to prepare a PC-Zn nanoparticle suspension following the steps of the previous examples with hyaluronic acid as a stabilizer.

S3-S4: Pretreated B16F10 tumor cells (2×10⁵ cells) were processed as described above to yield tumor cells (C4) encapsulated with the PC-Zn nanocoating.

### Example 5: Preparation of Tumor Cells (C5) Encapsulated with PC-Zn Nanocoating Without Stabilizer

S1: A proanthocyanidin (PC) solution (200 μL, 150 mg/mL) was dispersed in 1 mL of physiological saline, followed by the addition of a zinc chloride (ZnCl₂) solution (40 μL, 150 mg/mL). The mixture was shaken at 100 rpm for 5 minutes to obtain a first solution.

S2: A first phosphate buffer (pH=5.0, 20 mM, 1 mL) was added to the first solution and shaken at 100 rpm for 5 min to obtain a PC-Zn nanoparticle suspension.

S3: Pretreated B16F10 tumor cells (2×10⁵ cells) were resuspended in 1 mL of physiological saline (0.9% sodium chloride solution). Subsequently, 400 μL of the PC-Zn nanoparticle suspension was added to the cell suspension, and the mixture was shaken at 200 rpm for 5 minutes to obtain a second solution.

S4: A second phosphate buffer (pH=8.0, 20 mM, 500 μL) was added to the second solution and shaken at 200 rpm for 5 min to yield tumor cells (C5) encapsulated with a nanocoating composed of PC-Zn nanoparticles.

### II. Performance Testing and Characterization

### Example 6: Characterization of Encapsulated Stem Cells (C1)

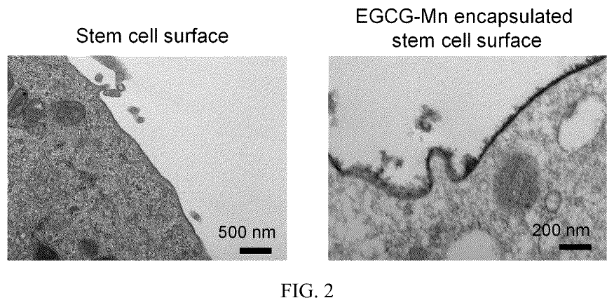

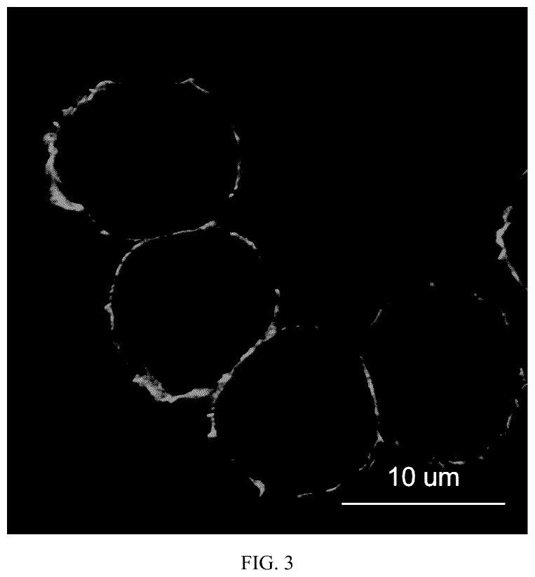

Cell surfaces were characterized using transmission electron microscopy (TEM). FIG. 2 illustrates the TEM cross-sections of original stem cells versus encapsulated stem cells (C1). The results confirm that the HA-embedded EGCG-Mn nanoparticles formed a dense nanocoating, whereas the original cells remained smooth, indicating complete encapsulation. Fluorescent probes (FITC-BSA) further confirmed the presence and loading of the EGCG-Mn nanocoating on the cell surface as shown in FIG. 3.

### Example 7: Analysis of Retention of Cellular Contents in Stem Cells

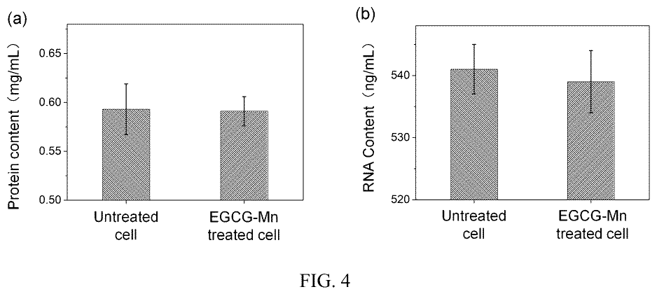

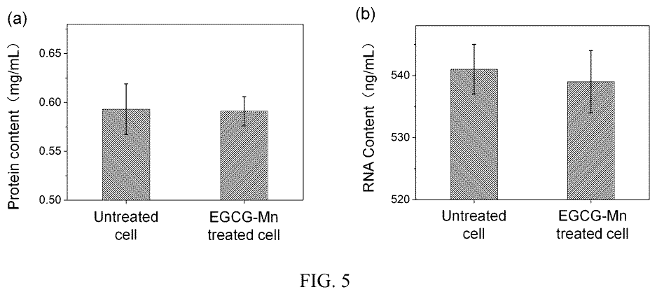

Untreated stem cells and encapsulated cells (C1) were subjected to hypotonic lysis and centrifuged at 300 g for 3 min. Gel electrophoresis of the sediment (FIG. 4) demonstrated that the protein content collected from inactivated C1 cells (Lane 3) was substantially identical to the original cells (Lane 1), whereas untreated cells showed significant loss (Lane 2). This confirms that the nanocoating effectively retains critical therapeutic factors such as TGF-β, IGF-1, and VEGF47. Quantitative analysis (FIG. 5) showed that the nanocoating reduced protein leakage from 50 μg/mL to 5 μg/mL and RNA leakage from 100 ng/mL to 10 ng/mL, representing a 90% reduction in leakage rate.

### Example 8: Characterization of Encapsulated E. coli Cells (C2)

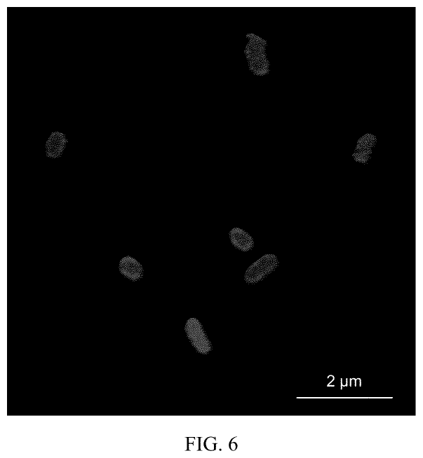

Specifically, after the encapsulation of E. coli cells with the EGCG-Mn nanocoating, bovine serum albumin labeled with a cyanine-based near-infrared fluorescent dye (Cy7-BSA) was added as a fluorescent probe. The experimental results are shown in FIG. 6. Confocal microscopy imaging revealed fluorescence surrounding the cells, thereby demonstrating the presence and successful formation of the EGCG-Mn nanocoating on the cell surface.

### Example 9: Retention of Endotoxins in Bacterial Cells

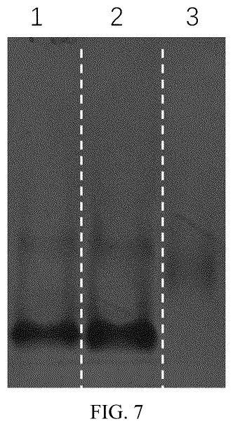

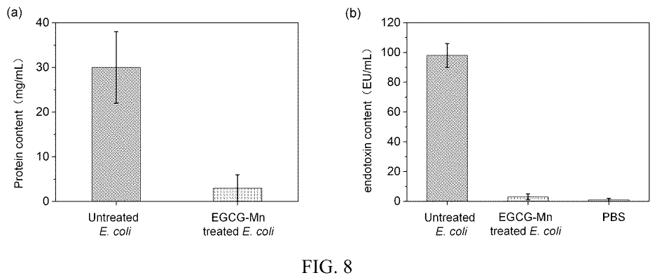

Encapsulated E. coli (C2) and untreated controls were lysed and analyzed 51. Electrophoresis (FIG. 7) showed that C2 cells (Lane 2) retained endotoxin protein levels identical to original cells (Lane 1). Supernatant testing (FIG. 8) revealed that while untreated cells released high levels of endotoxins and proteins, the levels in the C2 supernatant were significantly lower. The endotoxin leakage was reduced by 90% (from 100 EU/mL to 10 EU/mL), significantly lowering safety risks for secondary processing such as vaccine preparation.

### Example 10: Evaluation of Cell Inactivation

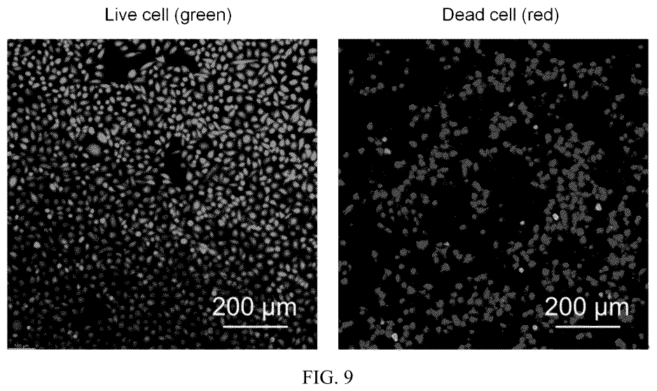

Tumor cells (C5) encapsulated with the PC-Zn nanocoating were tested using Calcein/PI staining. As shown in FIG. 9, treated cells completely lost their ability to proliferate within 12 hours, with cell viability approaching zero, demonstrating the highly effective inactivation achieved by the present invention.

What is claimed is:

1. A nanocoating for retaining cellular contents, comprising:
raw materials including metal ions and plant polyphenols;
wherein said metal ions and plant polyphenols are configured to form metal-polyphenol nanoparticles under acidic conditions; and
wherein said metal-polyphenol nanoparticles, after binding with a cell, form a nanocoating adhering to a surface of said cell under alkaline conditions.

2. The nanocoating of claim 1, further comprising a stabilizer, wherein said stabilizer is configured to react with the metal ions and plant polyphenols under acidic conditions to form the metal-polyphenol nanoparticles.

3. The nanocoating of claim 2, wherein the stabilizer is selected from the group consisting of hyaluronic acid, chitosan, carboxymethyl cellulose, alginic acid, and alginate salts.

4. The nanocoating of claim 1, wherein the acidic conditions comprise a pH value of 3.5 to 5.5, and the alkaline conditions comprise a pH value of 7.0 to 9.0.

5. The nanocoating of claim 1, wherein a molar ratio of the metal ions to the plant polyphenols is from 4:1 to 2:1.

6. The nanocoating of claim 1, wherein the metal-polyphenol nanoparticles have a diameter of 5 nm to 50 nm.

7. A method for encapsulating cells to retain cellular contents, comprising the steps of:
mixing a plant polyphenol solution and a metal ion solution to obtain a first solution;
adding a stabilizer to the first solution and adjusting the first solution to an acidic pH to obtain a metal-polyphenol nanoparticle suspension; and
adding the metal-polyphenol nanoparticle suspension to a cell suspension containing cells to be encapsulated to obtain a second solution, and adjusting the second solution to an alkaline pH to form a nanocoating on a surface of said cells.

8. The method of claim 7, wherein a molar ratio of the metal ions in the metal ion solution to the plant polyphenols in the plant polyphenol solution is from 4:1 to 2:1.

9. An application of the nanocoating of claim 1 for encapsulating stem cells or tumor cells, wherein the nanocoating is configured to prevent leakage of growth factors and cytokines from the stem cells, or to prevent leakage of proteins and RNA from the tumor cells, to maintain structural integrity of the cells.

10. An application of the nanocoating of claim 1 for encapsulating bacterial cells, wherein the nanocoating is configured to prevent leakage of biological macromolecules and metabolites from within the bacterial cells.
